(19) United States
(12) Patent Application Publication
(10) Pub. No.: US 2007/0238881 A1
(43) Pub. Date: Oct. 11, 2007

(54) Process for the preparation of tazarotene intermediates and use thereof for the preparation of tazarotene

(75) Inventors: Bobba Venkata Siva KUMAR, Maharashtra (IN); Pravin Balachandra KULKARNI, Maharashtra (IN); Changdev Namdev RAUT, Maharashtra (IN); Sanjay Dashrath VAIDYA, Maharashtra (IN); Nitin Sharad Chandra PRADHAN, Maharashtra (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Mumbai (IN)

(21) Appl. No.: 11/784,496

(22) Filed: Apr. 6, 2007

(30) Foreign Application Priority Data
Apr. 7, 2006 (IN) 559/MUM/2006

(51) Int. Cl. C07D 409/02

(52) U.S. Cl. 546/280.1

(57) ABSTRACT

The present invention provides a novel intermediate of ethyl 6-[2-(4,4-dimethylthiochroman-6-yl)ethynyl] nicotinate or a pharmaceutically acceptable salt thereof and a process for its preparation. The present invention also provides for the preparation of ethyl 6-[2-(4,4-dimethylthiochroman-6-yl)ethynyl] nicotinate of Formula I or a pharmaceutically acceptable salt thereof using the intermediate.

### PRIORITY

This application claims the benefit under 35 U.S.C. §119 to Indian Provisional Application 559/MUM/2006, filed on Apr. 7, 2006, and entitled “A PROCESS FOR THE PREPARATION OF TAZAROTENE INTERMEDIATES AND USE THEREOF FOR THE PREPARATION OF TAZAROTENE”, the contents of which are incorporated by reference herein.

### BACKGROUND OF THE INVENTION

1. Technical Field

The present invention generally relates to a process for the preparation of intermediates of tazarotene and their use in the preparation of tazarotene.

2. Description of the Related Art

Tazarotene, also known as ethyl 6-[2-(4,4-dimethylthiochroman-6-yl)-ethynyl] nicotinate, can be represented by the structure of general Formula I: 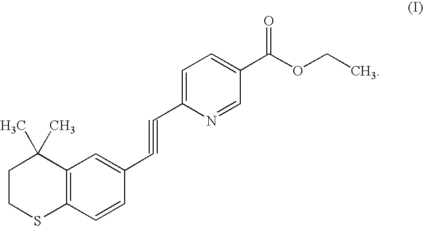
Tazarotene is a member of the acetylenic class of retinoids and is a prodrug that is converted to its active drug form, known as AGN 190299, in most biological systems by rapid deesterificaion of the cognate carboxylic acid of tazarotene. AGN 190299 binds to all three members of the retinoic acid receptor (RAR) family: RARα, RARβ, RARγ. AGN 190299 shows relative selectivity for the RARβ and RARγ and may modify gene expression. Tazarotene is ordinarily used in the treatment of psoriasis and is commercially available under the trade name Tazorace®. See, e.g., The Merck Index, Thirteenth Edition, 2001, p. 1621, monograph 9170.

It would be desirable to provide a novel process for preparing intermediates of tazarotene and their use in the preparation of tazarotene in a convenient and cost efficient manner and on a commercial scale.

### SUMMARY OF THE INVENTION

In accordance with a first embodiment of the present invention, a compound of Formula IV, 6-[2-(4,4-dimethylthiochroman-6-yl)ethynyl] nicotinonitrile and its N-oxide or a pharmaceutically acceptable salt thereof, is provided: 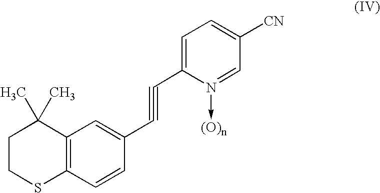
wherein n is 0 or 1.

In accordance with a second embodiment of the present invention, a process for the preparation of a compound of Formula IV or a pharmaceutically acceptable salt thereof is provided comprising:
(a) reacting 4,4-dimethyl-6-ethynylthiochroman of Formula II: 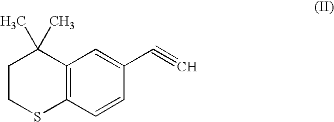
   with 6-halonicotinonitrile of Formula III: 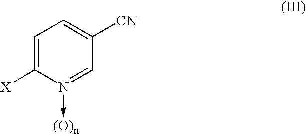
   wherein X is a halogen and n is 0 or 1, in the presence of a base, a transition metal catalyst and in a polar aprotic solvent to form a compound of Formula IV.

In accordance with a third embodiment of the present invention, a process for the preparation of ethyl 6-[2-(4,4-dimethylthiochroman-6-yl)ethynyl] nicotinate, a compound of Formula I, is provided: 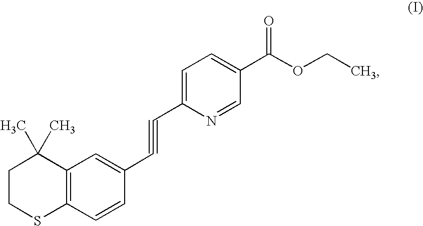
the process comprising the step of converting a compound of Formula IV to ethyl 6-[2-(4,4-dimethylthiochroman-6-yl)ethynyl] nicotinate of Formula I.

In accordance with a fourth embodiment of the present invention, a process for the preparation of ethyl 6-[2-(4,4-dimethylthiochroman-6-yl)ethynyl] nicotinate, a compound of Formula I is provided: 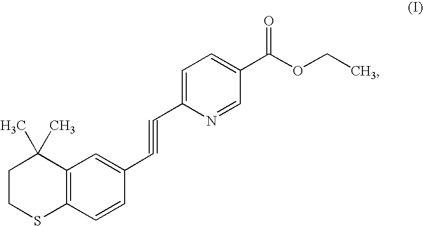
the process comprising;
(a) reacting 4,4-dimethyl-6-ethynylthiochroman of Formula II: 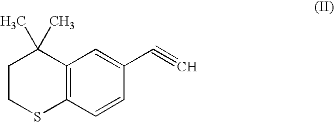
   with 6-halonicotinonitrile of Formula III: 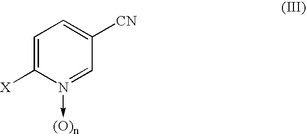
   wherein X is a halogen and n is 0 or 1, in the presence of a base, a transition metal catalyst and in a polar aprotic solvent to form a compound of Formula IV: 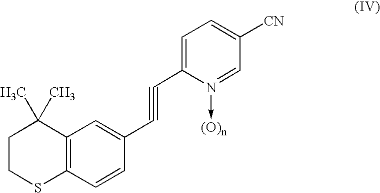
   wherein n is 0 or 1; and
(b) converting the compound of Formula IV to ethyl 6-[2-(4,4-dimethylthiochroman-6-yl)ethynyl] nicotinate of Formula I.

In accordance with a fifth embodiment of the present invention, a pharmaceutical composition is provided comprising tazarotene or a pharmaceutically acceptable salt thereof prepared by the processes of the present invention.

### DETAILED DESCRIPTION OF THE PREFERRED EMBODIMENTS

One embodiment of the present invention provides an intermediate of tazarotene of Formula IV and a process for its preparation: 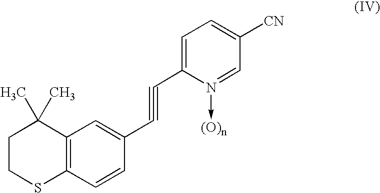
wherein n is 0 or 1. In general, a process for the preparation of the intermediate of Formula IV involves a coupling reaction.

In one embodiment, the process for preparing a compound of Formula I, 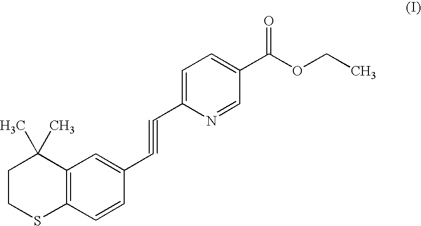
includes at least a coupling reaction between 4,4-dimethyl-6-ethynylthiochroman compound of Formula II: 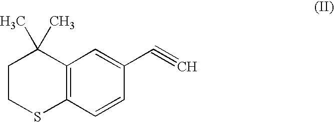
with 6-halonicotinonitrile of Formula III: 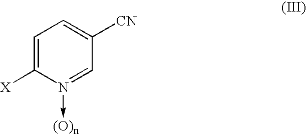
wherein X is a halogen such as Cl, Br or I, and n is 0 or 1, in the presence of a base, a transition metal catalyst and a polar aprotic solvent; to form a compound of Formula IV: 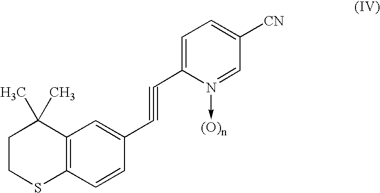
wherein n is 0 or 1.

The compounds of Formulae II and III are known in the art and can be prepared by any known method, for example, a compound of Formula II is disclosed in U.S. Pat. No. 5,023,341 and compound of Formula III is disclosed in European Patent No. EP 0619307A1, the contents of each of which are incorporated herein by reference.

A suitable base for use herein may be, for example, an organic base such as a primary, secondary or tertiary amine. Representative examples of such amines include, but are not limited to, triethylamine, tributylamine, diisopropylethylamine, diethylamine, N-methylmorpholine, pyridine, 4-(N,N-dimethylamino)pyridine, N,N-dimethylaniline, N,N-diethylaniline, 1,5-diazabicyclo[4.3.0]nona-5-ene, 1,4-diazabicyclo[2.2.2]octane (DABCO), 1,8-diazabicyclo[5.4.0]undec-7-ene (DBU) and the like and mixtures thereof. Alternatively, an inorganic base may be used and includes an alkali metal carbonate such as lithium carbonate, sodium carbonate, potassium carbonate and the like; alkali metal bicarbonate such as lithium bicarbonate, sodium bicarbonate, potassium bicarbonate and the like; alkali metal hydride such as lithium hydride, sodium hydride, potassium hydride and the like; alkali metal hydroxide such as lithium hydroxide, sodium hydroxide, potassium hydroxide and the like; alkali metal alkoxide such as lithium methoxide, sodium methoxide, sodium ethoxide, potassium t-butoxide and the like; and mixtures thereof. The organic amines (particularly triethylamine) are preferred.

The transition metal catalyst may be in the form of a salt or a complex with organic ligands. Particularly suitable metal catalysts are, for example, the Group VIII metals, such as Pd(0) complexes or a Pd(II) salt. However, the palladium catalyst used is not particularly limited provided that it is usually used for the Sonogashira coupling reaction. The ligands may be selected from, for example, phosphorus-containing ligands, such as triphenylphosphine (PPh₃) and 1,2-bis(diphenylphosphino)ethane (DPPE). Non-limiting examples of the transition metal catalysts include palladium salts such as palladium acetate, palladium chloride or palladium carbonate; and palladium complexes such as bis(triphenylphosphine) palladium (II) chloride (Pd[P(C₆H₅)₃]₂Cl₂) and palladium (0) based catalysts, such as Pd Cl₂(RCN)₂, wherein R is phenyl or methyl and mixtures thereof.

The reaction is advantageously carried out in a polar aprotic solvent. Suitable polar aprotic solvents include, but are not limited to, nitrites such as acetonitrile, isobutyronitrile and the like; dioxane, amides such as formamide, dimethylformamide, dimethylacetamide, hexamethylphosphoric triamide and the like; sulfoxides such as dimethyl sulfoxide, sulfolane and the like; as well as other polar aprotic solvents and mixtures thereof. Preferably, the polar aprotic solvent is an amide or sulfoxide with dimethyl sulfoxide, dimethyl form amide and dimethyl acetamide being more preferred. In a preferred embodiment of the present invention, the polar aprotic solvent is dimethyl sulfoxide. Generally, the amount of polar aprotic solvent employed in the coupling reaction can range from about 5 volumes to about 15 volumes and preferably from about 7 volumes to about 10 volumes.

The reaction can be carried out in the presence of a cuprous halide. The cuprous halide for use herein includes, but is not limited to, cuprous fluoride, cuprous chloride, cuprous bromide, cuprous iodide and the like and mixtures thereof. In a preferred embodiment of the process of the present invention, the cuprous halide is cuprous iodide.

The reaction temperature and time period for coupling the compounds of Formulae II and III will ordinarily depend on the starting compounds, the base and the solvent employed in the reaction. Generally, the reaction can be carried out at a temperature of from about 20° C. to about 200° C. for about 5 minutes to about 48 hours and preferably from about 15 minutes to about 24 hours. The reaction is advantageously conducted under an inert atmosphere such as nitrogen. Generally, a solution containing the transition metal catalyst and solvent may first be heated to a temperature ranging from about 130° C. to about 150° C. and preferably from about 140° C. to about 145° C. under a nitrogen atmosphere. As one skilled in the art will readily appreciate, the transition metal catalyst may be formed in situ by adding the salt with the organic ligands to the solution.

Next, a solution of 4,4-dimethyl-6-ethynylthiochroman (a compound of Formula II), 6-halonicotinonitrile (a compound of Formula III), base (e.g., triethanolamine) and cuprous halide are mixed separately and then added to the solution containing the transitional metal catalyst and solvent. The reaction mixture may then be heated to a temperature ranging from about 80° C. to about 100° C., and preferably to a temperature from about 95° C. to about 100° C., and stirred for about 2 to about 4 hours, and preferably about 3 hours.

Another aspect of the present invention provides a process for the preparation of ethyl 6-[2-(4,4-dimethylthiochroman-6-yl)ethynyl] nicotinate, a compound of Formula I

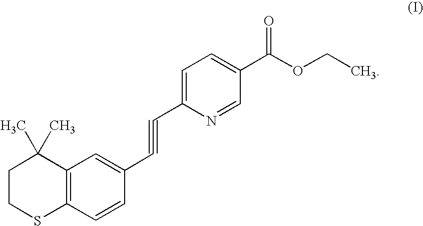

Generally, a compound of Formula I can be obtained by converting a compound of Formula IV to ethyl 6-[2-(4,4-dimethylthiochroman-6-yl)ethynyl] nicotinate of Formula I.

In one aspect of the present invention, a compound of Formula IV is hydrolyzed with an aqueous alkaline solution such as sodium hydroxide-containing solution, potassium hydroxide-containing solution, lithium hydroxide-containing solution and mixtures thereof; an alkali such as sodium hydroxide, potassium hydroxide, lithium hydroxide and mixtures thereof in the presence of one or more organic solvents such as an alcohol, ketone, ether and the like and mixtures thereof; an aqueous acidic solution such as hydrochloric acid-containing solution, sulfuric acid-containing solution, methanesulfonic acid-containing solution and the like and mixtures thereof; or an acid such hydrochloric acid, sulfuric acid, methanesulfonic acid and the like and mixtures thereof in the presence of one or more organic solvents such as alcohols, ketones, ethers and mixtures thereof to obtain 6-[(4,4-dimethyl-3,4-dihydro-2H-thiochromen-6-yl)ethynyl]nicotinamide (a compound of Formula IVa). Next, the resulting 6-[(4,4-dimethyl-3,4-dihydro-2H-thiochromen-6-yl)ethynyl]nicotinamide (a compound of Formula IVa) is optionally isolated and/or further hydrolyzed to obtain tazarotenic acid or its N-oxide (a compound of Formula V or a pharmaceutically acceptable salt thereof).

In another aspect of the present invention, a process for the preparation of ethyl 6-[2-(4,4-dimethylthiochroman-6-yl)ethynyl] nicotinate (a compound of Formula I) includes esterifying a compound of Formula V, wherein n is 0, or its pharmaceutically acceptable salts. The esterification of the compound of Formula V, wherein n is 0 can be carried out in the presence of an acid, a triethyl orthoester or ethanol in a suitable solvent. A useful acid includes, but is not limited to, sulfuric acid, hydrochloric acid, methanesulfonic acid etc. and the like and mixtures thereof. Useful triethyl orthoesters include, but are not limited to, triethyl orthoacetate, triethyl orthoformate and the like and mixtures thereof.

Suitable solvents include aliphatic hydrocarbon solvents such as heptane, decane, undacane etc and the like and mixture thereof, aromatic hydrocarbon solvents such as toluene, xylenes and the like and mixture thereof, non-aromatic hydrocarbon solvents such as dimethylsulfoxide, dimethyl formamide etc and the like and mixture thereof. Preferably esterification can be carried out in the presence of triethyl orthoacetate and toluene. The reaction may be advantageously carried out at about 20° C. to about 200° C., preferably at about 80° C. to about 160° C., more preferably at about 100° C. to about 140° C., and most preferably at about 110C. to about 120° C.

In another aspect of the present invention, a process for the preparation of 6-[2-(4,4-dimethylthiochroman-6-yl)ethynyl] nicotinic acid (a compound of Formula V wherein n is 0) includes reducing the N-oxide of the compound of Formula V wherein n is 1. The reduction of the compound of Formula V wherein n is 1 can be carried out in the presence of suitable reducing agent in a suitable solvent or in a neat condition. A useful reducing agent includes, but is not limited to, phosphorous oxy chloride, phosphorus pentachloride, trifluoroacetic acid anhydride and the like and mixtures thereof. Suitable solvents include, but are not limited to, chlorinated solvents such as methylene chloride, dichloroethane, perchloroethylene, chlorobenzane and the like and mixtures thereof.

The 6-[2-(4,4-dimethylthiochroman-6-yl)ethynyl] nicotinic acid thus obtained (a compound of Formula V wherein n is 0) is converted into ethyl 6-[2-(4,4-dimethylthiochroman-6-yl)ethynyl] nicotinate (a compound of Formula I) as described above.

In another aspect of the present invention, tazarotene thus obtained may be purified. For example, an inorganic acid may be added to the reaction mixture prior to any isolation or following isolation of tazarotene to provide a salt of the tazarotene. Examples of suitable inorganic acids include, but are not limited to, hydrobromic acid, hydrochloric acid, sulfuric acid, perchloric acid, phosphoric acid and the like, as well as solutions of the inorganic acid, e.g., in an acetate such as ethyl acetate, with hydrochloric acid being preferred. By adding the inorganic acid to the reaction mixture, a salt of the tazarotene is advantageously formed. If desired, the inorganic acid can be added as a solution further containing a suitable solvent such as, for example, ethyl acetate. The salt obtained can then be dissolved in a second solvent and an inorganic base may be added such that the tazarotene can be isolated by conventional techniques. This may allow for a higher yield of the resulting tazarotene from the salt compound, e.g., a yield of at least about 65% and preferably at least about 80% with desired purity.

The second solvent for use herein includes, but is not limited to, aromatic hydrocarbon solvents such as toluene, xylene and the like and mixtures thereof; ketones such as methyl isobutyl ketone and the like and mixtures thereof; acetates such as methyl acetate, t-butyl acetate and the like and mixtures thereof, alcohols such as methanol, ethanol, N-butanol and the like and mixtures thereof.

A suitable inorganic base for use herein includes, but is not limited to, alkali metal carbonates such as potassium carbonate, sodium carbonate, and the like; alkali metal bicarbonates such as potassium bicarbonate and the like; alkali metal hydroxides such as sodium hydroxide, potassium hydroxide and the like and mixtures thereof.

In another aspect of the present invention, tazarotene thus obtained may be further purified by dissolving the tazarotene in a C₁₋₄ alcohol such as methanol, ethanol, propanol, butanol and the like and mixture thereof, with ethanol being preferred. The solvent may be heated to obtain a solution at a temperature of from about ambient temperature to about reflux temperature. The reaction solution may be cooled at a temperature from about 20° C. or less such that the tazarotene can be isolated by conventional techniques. This may allow for a high purity level of the resulting tazarotene from the crude tazarotene, e.g., a purity of at least about 95% preferably at least about 98% and more preferably at least about 99.5%.

In one preferred embodiment of the present invention, tazarotene is prepared according to Scheme I:

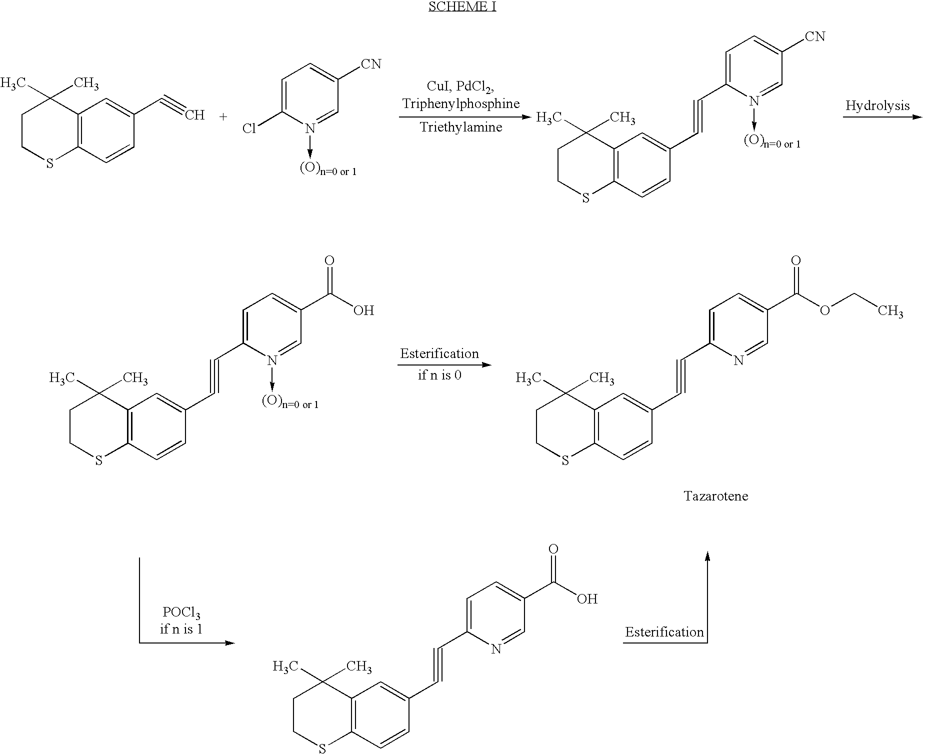

By performing the processes of the present invention, substantially pure tazarotene can be obtained with a degree of purity as determined by HPLC greater than or equal to about 98%, preferably greater than or equal to about 99% and most preferably greater than or equal to about 99.5% as compared to the crude product. Also, the content of free tazarotene in the final product as determined by HPLC can be at a level of less than about 2%, preferably less than about 1% and more preferably less than about 0.5% as compared to the crude product. Moreover, the substantially pure tazarotene may be obtained substantially free of any unknown impurity, e.g., a content of less than about 0.1% of impurities. The crude tazarotene for use herein can have purity as determined by HPLC which can ordinarily vary in the range of from about 92% to less than 98%.

Another aspect of the present invention is directed to a pharmaceutical composition containing at least the substantially pure tazarotene disclosed herein and at least one pharmaceutically acceptable excipient. Such pharmaceutical compositions may be administered to a mammalian patient in any dosage form, e.g., liquid, powder, elixir, injectabe solution, etc.

In one embodiment, the tazarotene or pharmaceutically acceptable salt thereof disclosed herein for use in the pharmaceutical compositions of the present invention can have a D₅₀ and D₉₀ particle size of less than about 400 microns, preferably less than about 200 microns, more preferably less than about 150 microns, still more preferably less than about 50 microns and most preferably less than about 15 microns. The particle sizes of the tazarotene or pharmaceutically acceptable salt thereof prepared according to the present invention can be obtained by any milling, grinding micronizing or other particle size reduction method known in the art to bring the solid state tazarotene or pharmaceutically acceptable salt thereof into any of the foregoing desired particle size range.

The dosage forms may contain the substantially pure tazarotene disclosed herein or, alternatively, may contain the substantially pure tazarotene as part of a composition. Whether administered in pure form or in a composition, the substantially pure tazarotene may be in any form, for example, compacted tablets, powder suspensions, capsules, and the like. The compositions of the present invention can be administered to humans and animals in such dosage forms as oral, rectal, parenteral (intravenous, intramuscular, or subcutaneous), intracistemal, intravaginal, intraperitoneal, local (powders, ointments or drops), ophthalmic, transdermal, or sublingual forms or as a buccal or nasal spray. Oral dosage forms include, burt are not limited to, pills, capsules, troches, sachets, suspensions, powders, lozenges, elixirs, tablets, capsules (including soft gel capsules), ovules, solutions, and the like which may contain flavoring or coloring agents, for immediate-, delayed-, modified-, or controlled-release such as sustained-, dual-, or pulsatile delivery applications. The substantially pure tazarotene disclosed herein also may be administered as suppositories, ophthalmic ointments and suspensions, and parenteral suspensions, which are administered by other routes. The most preferred route of administration of the substantially pure tazarotene of the present invention is oral.

The active ingredient of the invention may also be administered via fast dispersing or fast dissolving dosage forms or in the form of high energy dispersion or as coated particles. Suitable pharmaceutical composition of the invention may be in coated or uncoated form as desired.

Tabletting compositions may have few or many components depending upon the tabletting method used, the release rate desired and other factors. For example, the compositions of the present invention may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols like mannitol and sorbitol, acrylate polymers and copolymers, as well as pectin, dextrin and gelatin.

Other excipients contemplated by the present invention include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

Capsule dosages will contain the solid composition within a capsule which may be coated with gelatin. Tablets and powders may also be coated with an enteric coating. The enteric-coated powder forms may have coatings comprising phthalic acid cellulose acetate, hydroxypropylmethyl cellulose phthalate, polyvinyl alcohol phthalate, carboxymethylethylcellulose, a copolymer of styrene and maleic acid, a copolymer of methacrylic acid and methyl methacrylate, and like materials, and if desired, they may be employed with suitable plasticizers and/or extending agents. A coated tablet may have a coating on the surface of the tablet or may be a tablet comprising a powder or granules with an enteric coating.

The following examples are provided to enable one skilled in the art to practice the invention and are merely illustrative of the invention. The examples should not be read as limiting the scope of the invention as defined in the claims.

### EXAMPLE 1

Preparation of 6-[2-(4,4-dimethylthiochroman-6-yl)ethynyl] nicotinonitrile.

Into a 500 ml 4 necked round bottom flask fitted with a mechanical stirrer and a reflux condenser was charged dimethyl sulfoxide (150 ml), PdCl₂ (0.180 g), and triphenyl phosphine (0.825 g) under nitrogen atmosphere. The temperature was raised to 140 to 145° C. and maintained for 30 to 35 minutes. The reaction mixture was cooled to room temperature and added to a mixture of 6-chloronicotinonitrile (3.5 g), 4,4-dimethyl-6-ethynylthiochroman (4.5 g) in dimethyl sulfoxide (50 ml). The reaction mixture was stirred for 20 to 25 minutes followed by the addition of cuprous iodide (0.325 g) and triethylamine (10 g). The temperature was slowly raised to 70 to 75° C. and maintained for 3 to 5 hours. After completion of the reaction, the reaction mass was cooled to room temperature and filtered to remove insolubles. Ethyl acetate (75 ml) was added to the filtrate and the organic layer was quenched in water (200 ml). The ethyl acetate layer was separated, washed with water and concentrated to obtain 6-[2-(4,4-Dimethylthiochroman-6-yl)ethynyl]nicotinonitrile (1.5 g).

The IR (KBr) showed the following absorptions: 2955 cm⁻¹ (C—H str), 2197 cm⁻¹ (CN str); 1H NMR (CDCl₃)-TMS as an internal standard shows the following signals δ 1.336 (6H, s), 1.995 (3H, t), 3.051 (3H, t), 7.089 (1H, d), 7.24 (1H, d), 7.6 (2H, m), 7.9(1H, dd) and 8.834 (1H, s). The CI/MS shows m/z 305 (M+).

### EXAMPLE 2

Preparation of 6-[2-(4,4-dimethylthiochroman-6-yl)ethynyl] nicotinic acid.

Into a 250 ml 4-necked round bottom flask fitted with a mechanical stirrer and reflux condenser, ethanol (41.3 ml), sodium hydroxide (5.22 g, 0.1305 M) and 6-(4,4-dimethylthiochroman-6-ylethynyl) nicotinonitrile (3.0 gm, 0.01 M) were mixed and heated to reflux temperature 85° C. to 95° C. After completion of the reaction, the solvents were distilled off; water was added followed by pH adjustement to neutral by adding 5% sodium carbonate solution. The precipitated solids were filtered, washed with water and dried to get 6-(4,4-dimethylthiochroman-6-ylethynyl) nicotinic acid (2.2 g).

### EXAMPLE 3

Preparation of 6-[2-(4,4-dimethylthiochroman-6-yl)ethynyl] nicotinic acid.

Into a 250 ml 4-necked round bottom flask fitted with a mechanical stirrer and reflux condenser, water (41.3 ml) and sulfuric acid (5.22 g, 0.0533 M) and 6-[2-(4,4-dimethylthiochroman -6-yl)ethynyl] nicotinonitrile (3.0 g, 0.01 M) were added and heated to 70° C. to 75° C. After completion of the reaction, it was cooled to 0-10° and the pH of the reaction mass was slowly adjusted to neutral by adding 10% sodium carbonate solution. The precipitated solids were filtered, washed with water and dried to get 6-(4,4-dimethylthiochroman-6-ylethynyl) nicotinic acid (2.2 g).

### EXAMPLE 4

Preparation of 6-[2-(4,4-dimethylthiochroman-6-yl)ethynyl] nicotinic acid hydrochloride.

Into a 250 ml 4-necked round bottom flask fitted with a mechanical stirrer and reflux condenser, ethanol (41.3 ml), sodium hydroxide (5.22 g, 0.1305 M) dissolved in 5 ml water and 6-[2-(4,4-dimethylthiochroman-6-yl)ethynyl) nicotinonitrile (3.0 g, 0.01 M) were mixed and heated to a reflux temperature. After completion of the reaction, the reaction mass was cooled to 20-25° C. and then further cooled to 0-10° C. followed by the addition of 50 ml of ethyl acetate hydrochloride solution (8-10%). The reaction mass was further stirred for 4 hours at room temperature and the precipitated solids were filtered to isolate 6-(4,4-dimethylthiochroman-6-ylethynyl) nicotinic acid hydrochloride (2.0 g).

### EXAMPLE 5

Preparation of 6-[2-(4,4-dimethylthiochroman-6-yl)ethynyl] nicotinic acid ethyl ester

Into a 250 ml 4-necked round bottom flask fitted with a mechanical stirrer and reflux condenser, 6-[2-(4,4-dimethylthiochroman-6-yl)ethynyl] nicotinic acid (3.0 g, 0.01 M) was dissolved in ethanol (50 ml) and concentrated sulfuric acid (1.0 g) was added and heated to reflux temperature. After completion of the reaction as determined by TLC, the solvents were distilled off; water was added followed by pH adjustement to neutral by adding 10% sodium carbonate solution slowly. The product was extracted with ethyl acetate (25 mlx3). The ethyl acetate layer was then washed with brine (15 mlx3). The organic layer was dried over sodium sulfate, and the solvent was distilled off to get 6-(4,4-dimethylthiochroman-6-ylethynyl) nicotinic acid ethyl ester (1.0 g).

### EXAMPLE 6

Preparation of 6-[2-(4,4-dimethylthiochroman-6-ylethynyl) nicotinic acid ethyl ester

Into a 500 ml 4 necked round bottom flask fitted with a mechanical stirrer and a reflux condenser was charged toluene (150 ml), triethyl orthoacetate (50 ml), and 6-[2-(4,4-dimethylthiochro -6-yl)ethynyl] nicotinic acid (5.0 g) under nitrogen atmosphere. The mass temperature was raised to reflux temperature 110° C. to 120° C. After completion of reaction it was cooled to room temperature and water was added. The organic layer was washed with brine solution, dried over anhydrous sodium sulfate and concentrated to get 6-(4, 4-dimethylthiochroman-6-ylethynyl) nicotinic acid ethyl ester (2.5 gm).

### EXAMPLE 7

Preparation of 6-[2-(4,4-dimethylthiochroman-6-yl)ethynyl] nicotinic acid ethyl ester

Into a 500 ml 4 necked round bottom flask fitted with a mechanical stirrer and a reflux condenser was charged toluene (150 ml), triethyl orthoacetate (50 ml), and 6-[2-(4,4-dimethylthiochro -6-yl)ethynyl] nicotinic acid hydrochloride (5.0 g) under nitrogen atmosphere. The mass temperature was raised to reflux temperature 11I0° C. to 120° C. After completion of reaction it was cooled to room temperature and water was added. The organic layer was washed with 5% sodium carbonate solution followed by brine solution, dried over anhydrous sodium sulfate and concentrated to get 6-[2-(4,4-dimethylthiochroman-6-yl)ethynyl] nicotinic acid ethyl ester (3.2 g).

### EXAMPLE 8

Preparation of 6-[2-(4,4-dimethylthiochroman-6-yl)ethynyl] nicotinic acid ethyl ester hydrochloride

Into a 500 ml 4 necked round bottom flask fitted with a mechanical stirrer and a reflux condenser was charged toluene (150 ml), triethyl orthoacetate (50 ml), and 6-[2-(4,4-dimethylthiochro -6-yl)ethynyl] nicotinic acid hydrochloride (5.0 g) under nitrogen atmosphere. The mass temperature was raised to reflux temperature 110° C. to 120° C. After completion of reaction it was cooled to room temperature and water was added. The organic layer was washed with 5% sodium carbonate solution followed by brine solution, dried over anhydrous sodium sulfate. Ethyl acetate hydrochloride (50 ml) was added slowly to the toluene solution at temperature 20° C. to 30° C. and stirred for 2 to 4 hours and filtered the product, washed with ethyl acetate to get 6-[2-(4,4-dimethylthiochroman-6-yl)ethynyl] nicotinic acid ethyl ester hydrochloride (2.5 g).

### EXAMPLE 9

Preparation of 6-[2-(4,4-dimethylthiochroman-6-ylethynyl) nicotinic acid ethyl ester.

Into a 500 ml 4 necked round bottom flask fitted with a mechanical stirrer was charged ethyl acetate (150 ml), sodium bicarbonate solution (5.0 g in 50 ml of water) and 6-[2-(4,4-dimethylthiochroman-6-yl)ethynyl] nicotinic acid ethyl ester hydrochloride (5.0 g) and stirred for 2 to 4 hours. The ethyl acetate layer was separated and washed with water (2×100 ml). The organic layer was dried over anhydrous sodium sulfate and concentrated to obtain 6-[2-(4,4-dimethylthiochroman-6-ylethynyl) nicotinic acid ethyl ester. (3.75 g)

### EXAMPLE 10

Purification of 6-[2-(4,4-dimethylthiochroman-6-ylethynyl) nicotinic acid ethyl ester.

Into a 500 ml 4 necked round bottom flask fitted with a mechanical stirrer and a reflux condenser was charged ethanol (25 ml) and 6-[2-(4,4-dimethylthiochroman-6-yl)ethynyl] nicotinic acid ethyl ester (5.0 g). The mass temperature was raised to reflux temperature and maintain for 10 to 20 min. The reaction mass was cooled to 25 to 30° C. and stirred for 1 to 2 hour. The precipitated solids were filtered, washed with ethanol and dried to obtain 6-[2-(4,4-dimethylthiochroman-6-yl)ethynyl] nicotinic acid ethyl ester (3.5 g).

### EXAMPLE 11

Preparation of phenyl-3-methylbut-2-enyl sulfide

Into a 5 L 4-neck round bottom flask, methanol (1400 ml) and thiophenol (200 g) were added under stirring at a temperature ranging from about 25° C. to about 35° C. Sodium hydroxide (powder LR grade) (73.60 g) and methanol (100 ml) were added to the mixture under stirring. The reaction mixture was left under a nitrogen atmosphere and stirred at room temperature (about 250° C. to about 300° C.) for an hour. Next, 1-bromo-3-methyl-2-butene (274 gm) was added to the reaction mixture and it was observed that the temperature rose to about 40° C. The reaction mixture was heated to reflux and maintained for about 12 hours. After completion of the reaction as determined by HPLC, the methanol was distilled out from the reaction mixture under vacuum at a temperature below 60° C. Ethylene dichloride (1500 ml) and water (1000 ml) were added to the residue. The organic layer was separated and washed with a 5% sodium hydroxide (600 ml) solution, and then water (3×600 ml) until the pH was about 7. The organic layer was then washed with a brine solution (700 ml). The ethylene dichloride was distilled out until the moisture content was less than 0.1%.

### EXAMPLE 12

Preparation of 4,4-dimethylthiochroman

Into a 5 L 4-neck round bottom flask, ethylene dichloride (1500 ml) was added to the phenyl-3-methylbut-2-enyl sulfide obtained in Example 11. Phosphorous pentoxide (200 g) was added to the reaction mixture at a temperature ranging from about 25° C. to about 35° C. under stirring. Next, ortho phosphoric acid (174 ml) was added carefully under nitrogen. The reaction mixture was heated to reflux, a temperature of about 80° C. to about 90° C. and maintained at that temperature for about 12 hours. After completion of the reaction as determined by HPLC, the reaction mass was cooled to a temperature ranging from about 25° C. to about 35° C. and water (2000 ml) was slowly added to the reaction mass. The organic layer was separated, and the aqueous layer was extracted with ethylene dichloride (2 Lx2). The organic layers were combined and washed with saturated sodium bicarbonate solution (2 LX2) and water (1.5 LX2) until the pH was about 7. This was followed by a washing with a brine solution (1.5 L). The ethylene dichloride layer was distilled out under reduced pressure below a temperature of about 70° C. until the moisture content was less than 0.1%. Ethylene dichloride (2 L) was added to the residue and taken for the next step without further purification

### EXAMPLE 13

Preparation of 4,4-dimethyl-6-acetylthiochroman

Into a 5 L 4-neck round bottom flask, ethylene dichloride (2 L) was added to the 4,4-dimethylthiochroman obtained in Example 12. The contents were stirred and cooled to a temperature of about −10C. Aluminum chloride (252 g) was slowly added to the reaction mixture. Acetyl chloride (152.7 g) was added at a temperature ranging from about −10° C. to about −5° C. over about 1.5 hours. After the addition, the reaction mixture was maintained at a temperature ranging from about −5° C. to about 0° C. for about 2 hours. The reaction was monitored by TLC. If the reaction is incomplete as determined by TLC, bring the reaction mixture to a temperature ranging from about 25° C. to about 35° C. under stirring for about 4 hours. The reaction mixture was quenched with ice (4.87 kg) and hydrochloric acid (1.63 L), and the reaction mass was stirred for about 30 minutes. Ethylene dichloride (2.5 L) was added to the reaction mass and the layers were separated. The aqueous layer was extracted with methylene dichloride (2×2 L). The organic layers were combined and washed with 5% sodium bicarbonate solution (2×2 L) and water (2×2 L) until the pH was about 7. This was followed by a washing with brine (1.5 L). The ethylene dichloride and methylene dichloride layer were distilled out under reduced pressure until the moisture content was less than about 0.1%. There was a residual volume of about 3 L.

### EXAMPLE 14

Preparation of 3-[4,4-dimethylthiochroman-6-yl]-3-chloro-2-propene-1-al

Into a 500 ml 4-necked round bottom flask fitted with a mechanical stirrer and a reflux condenser, 6-acetyl-4,4-dimethylthio-chroman (22 g) and dimethylformamide (38 ml) were added at a temperature in the range of from about 35° C. to about 95° C. under stirring. The reaction mixture was then cooled to a temperature in the range of from about −5° C. to about 0° C. Phosphorus oxychloride (17.2 g) was added to the reaction mixture dropwise over about 30 minutes. Following the addition of the phosphorous oxychloride, the reaction mixture was maintained at a temperature in the range of from about 10° C. to about 15° C. for about 8 hours to about 10 hours. After completion of the reaction as determined by TLC, the reaction mixture was added to cold water (100 ml) at a temperature of from about 0° C. to about 5° C. containing sodium acetate (25 g). The aqueous layer was extracted with dichloromethane (DCM) (200 ml×3). The organic layer was washed with demineralized water (100 ml×3) until it became neutral.

The DCM layer was concentrated on a rotavapor bath at a temperature in the range of from about 25° C. to about 30° C. under plant vacuum until no more drops were observed. The resulting residual oil was purified by flash chromatography with petroleum ether and ethyl acetate (9:1 mixture) resulting in a pale yellow oil, weighing about 22 g, yield of about 82%, purity of about 98% (HPLC).

### EXAMPLE 15

Preparation of 4,4-dimethyl-6-ethynylthiochroman

Into a 250 ml 4-necked round bottom flask fitted with a mechanical stirrer and reflux condenser, water (41.3 ml) and sodium hydroxide (5.22 g, 0.1305 M) were added and heated to a temperature in the range of from about 80° C. to about 90° C. The reaction mixture was stirred, and a solution of 3-[4,4-dimethylthiochroman-6-yl]-3-chloro-2-propene-1-al (3.0 gm, 0.0113 M) was added drop wise in 1,4-dioxane (52.2 ml) under vigorous stirring. The reaction mixture was maintained at a temperature in the range of from about 80° C. to about 90° C. for about 2 hours. After completion of the reaction as determined by TLC, the solvents were distilled off and the product was extracted with ether (15 ml×3). The ether layer was washed with brine (15 ml×3). The organic layer was dried over sodium sulfate, and the solvent was distilled off to get an oily residue. The resulting crude oil was distilled under high vacuum and the vapors were collected at a temperature of about 126° C./0.2 mm as the main product. The main fraction appeared as a red viscous oil, which upon standing crystallized. The product showed a net weight of about 2.00 g, a yield of about 87.68%; a m.p. in the range of from about 69° C. to about 72° C., and a purity of about 98% (HPLC).

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

What is claimed is:

1. A process for the preparation of ethyl 6-[2-(4,4-dimethylthiochroman-6-yl)ethynyl] nicotinate of Formula I or a pharmaceutically acceptable salt thereof: 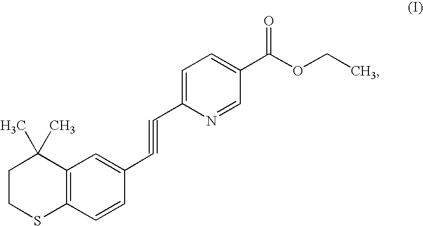
the process comprising converting a compound of Formula IV: 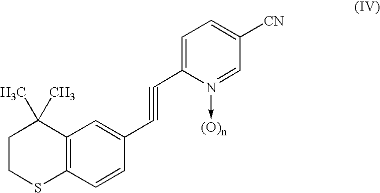
wherein n is 0 or 1 to the compound of Formula I.

2. The process of claim 1, comprising (a) reacting 4,4-dimethyl-6-ethynylthiochroman of Formula II: 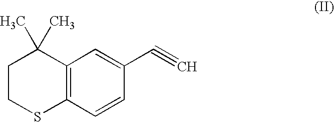
with a 6-halonicotinonitrile of Formula III: 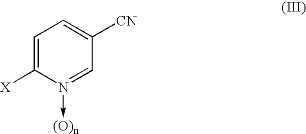
wherein X is a halogen and n is 0 or 1, in the presence of a base, a transition metal catalyst and in a polar aprotic solvent to form a compound of Formula IV and (b) converting the compound of Formula IV to the compound of Formula I.

3. The process of claim 1, wherein the step of converting comprises subjecting the compound of Formula IV to a first hydrolysis and then esterification.

4. The process of claim 3, comprising
subjecting the compound of Formula IV to first hydrolysis to obtain 6-[(4,4-dimethyl-3,4-dihydro-2H-thiochromen-6-yl)ethynyl]nicotinamide of Formula IVa: 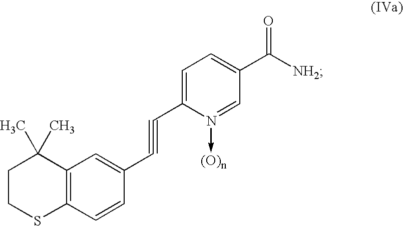
subjecting the compound of Formula IVa to a second hydrolysis to obtain a compound of Formula V or a pharmaceutically acceptable acid salt thereof: 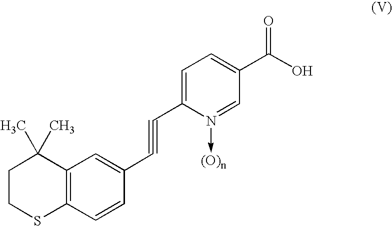
esterifying the compound of Formula V or a pharmaceutically acceptable acid salt thereof to esterification when n is 0 to obtain a compound of Formula I or a pharmaceutically acceptable salt thereof or,
reducing the N-oxide of the compound of Formula V when n is 1 and esterifying the reduced compound to obtain the compound of Formula I or a pharmaceutically acceptable acid salt thereof.

5. The process of claim 4, wherein the compound of Formula IV is subjected to a first hydrolysis using an acid selected from the group consisting of hydrochloric acid, sulfuric acid, methane sulfonic acid and mixtures thereof.

6. The process of claim 4, wherein the compound of Formula IV is subjected to a first hydrolysis using an alkali metal hydroxide selected from the group consisting of sodium hydroxide, potassium hydroxide, lithium hydroxide and mixtures thereof.

7. The process of claim 2, wherein the base is selected from the group consisting of an alkali metal carbonate, alkali metal bicarbonate, alkali metal hydride, alkali metal hydroxide, alkali metal alkoxide, organic amine and mixtures thereof.

8. The process of claim 2, wherein the base is an organic amine selected from the group consisting of triethylamine, tributylamine, diethylamine, diisopropylethylamine, N-methylmorpholine, pyridine, 4-(N,N-dimethylamino)pyridine, N,N-dimethylaniline, N,N-diethylaniline, 1,5-diazabicyclo[4.3.0]nona-5-ene, 1,4-diazabicyclo[2.2.2]octane, 1,8-diazabicyclo [5.4.0]undec-7-ene and mixtures thereof.

9. The process of claim 2, wherein the transition metal catalyst is a palladium catalyst.

10. The process of claim 9, wherein the palladium catalyst is selected from the group consisting of palladium acetate, palladium chloride, palladium carbonate, bis(triphenylphosphine) palladium (II) chloride and mixtures thereof.

11. The process of claim 2, wherein the polar aprotic solvent is selected from the group consisting of a nitrile, an amide, a sulfoxide and mixtures thereof.

12. The process of claim 11, wherein the polar aprotic solvent is selected from the group consisting of dimethyl sulfoxide, dimethyl formamide, dimethyl acetamide and mixtures thereof.

13. The process of claim 2, wherein the polar aprotic solvent is present in an amount of about 5 volumes to about 15 volumes.

14. The process of claim 2, wherein the polar aprotic solvent is present in an amount of about 7 volumes to about 10 volumes.

15. The process of claim 2, further comprising a cuprous halide selected from the group consisting of cuprous fluoride, cuprous chloride, cuprous bromide, cuprous iodide and mixtures thereof.

16. The process of claim 2, wherein the reaction is carried out at a temperature of about 20° C. to about 200° C.

17. A compound of Formula IV or a pharmaceutically acceptable salt thereof: 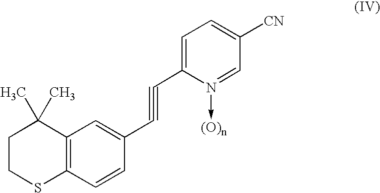
wherein n is 0 or 1.

18. The compound of claim 17, wherein n is 0.

19. A process for the preparation of a compound of Formula IV or a pharmaceutically acceptable salt thereof, the process comprising:
(a) reacting 4,4-dimethyl-6-ethynylthiochroman of Formula II: 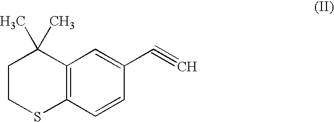
with a 6-halonicotinonitrile of Formula III: 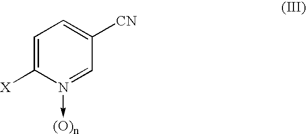
wherein X is a halogen and n is 0 or 1, in the presence of a base, a transition metal catalyst and a polar aprotic solvent; to form a compound of Formula IV.

20. The process of claim 1, further comprising recrystallizing the compound of Formula I with a C₁₋₄ alcohol.
